(19) United States
(12) Patent Application Publication
(10) Pub. No.: US 2012/0122197 A1
(43) Pub. Date: May 17, 2012

(54) INKJET REAGENT DEPOSITION FOR BIOSENSOR MANUFACTURING

(76) Inventor: Abner David Jospeh, Carmel, IN (US)

(21) Appl. No.: 12/944,817

(22) Filed: Nov. 12, 2010

(51) Int. Cl. C12M 1/00; B05D 3/00; B32B 37/14; B05D 3/06; B32B 37/02; B05D 5/12; B05D 3/02

(52) U.S. Cl. 435/283.1; 427/58; 427/559; 156/60

(57) ABSTRACT

A technique for producing a biosensor includes inkjet printing a reagent onto electrodes of the biosensor. The ink has been specially formulated to allow the reagent to be printed using inkjet printing while at the same time produce commercially viable biosensor. The inkjet printing of the reagent allows for different inkjet patterns to be produced as well as facilitates quick change over between various products. For example, the technique allows the reagent and electrode to be formed on opposite sides of a substrate. In another example, the reagent can be layered such that incompatible reagents can be separated by a barrier layer. The electrodes for the biosensor can also be inkjet printed such that most of the biosensor can be produced using inkjet technology.

### BACKGROUND

Home diagnostic testing has become very popular in recent years. With its widespread adoption, there has been increased price pressures on manufacturers of home diagnostic testing equipment. One component acutely affected by this price pressure is disposable biosensors, commonly referred to as test strips. While it is desirable for test strips to be inexpensive, they also have to be accurate, and as such require tightly controlled manufacturing processes. For example, the reagent used to analyze the body fluid sample can be quite expensive. At the same time, the reagent has to be precisely applied in a tightly controlled environment to ensure accurate test results. For instance, even small variances in the coating thickness of the reagent can adversely affect accuracy. Typical commercial reagent deposition techniques, such as slot-die coating and drop deposition, tend to be wasteful and can significantly limit the line speeds for producing the test strips. These traditional reagent deposition techniques are also not flexible enough so as to readily adapt to changes in layout of the test strip.

Thus, there is a need for improvement in this field.

### SUMMARY

Based on the limitations inherent to common slot-die coating and drop deposition techniques for applying reagents to the test strip, it was found that depositing the reagent through an inkjet printing technique could overcome these issues found in the traditional reagent deposition techniques. While some have suggested, in passing, that an inkjet printing could be used to apply reagent, usually in a long laundry list of other unrelated deposition techniques, there has been no inkjet printing technique that has been proposed that produces commercially viable biosensors. Inkjet printing requires a more robust formulation for the reagent so as to minimize impact on the activity of the enzymes. The inventors had to overcome a large number of significant and unforeseen obstacles in order to manufacture commercially viable biosensors using inkjet printing techniques for the reagent.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a block diagram of a system for manufacturing a biosensor using an inkjet printing technique.

FIG. 2 is a top view of the stages in which the electrodes and reagent are deposited on a substrate using the inkjet printing technique.

FIG. 3 is an enlarged top view of a reagent printhead inkjet printing reagent onto the substrate and electrodes.

FIG. 4 is a top view of an alternative embodiment in which the electrodes and reagent are inkjet printed in a direction that is generally aligned with a conveying path of the substrate.

FIG. 5 is an enlarged top view of an another embodiment in which the reagent printhead prints the reagent with alternating patterns.

FIG. 6 is an enlarged view of a reagent pattern that has two different reagent zones.

FIG. 7 is an enlarged view of a reagent pattern in which the reagent zones are printed longitudinally along the electrodes.

FIG. 8 is an enlarged view of a reagent pattern in which the reagent zones are spaced apart and have different shapes.

FIG. 9 is a diagram of a biosensor manufacturing process in which the electrodes and reagent are deposited on opposing sides of the substrate through inkjet printing techniques.

FIGS. 10, 11, and 12 are sequential end views of the biosensor when the capillary channel is formed.

### DETAILED DESCRIPTION

For the purposes of promoting an understanding of the principles of the invention, reference will now be made to the specific embodiments illustrated herein and specific language will be used to describe the same. It will nevertheless be understood that no limitation of the scope of the invention is thereby intended. Any alterations and further modifications in the described processes or devices and any further applications of the principles of the invention as described herein, are contemplated as would normally occur to one skilled in the art to which the invention relates. Preferred embodiments of the invention are subject of the dependent claims.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

As noted before, the traditional approaches for applying reagent to biosensors, such as traditional slot-die coating and screen or rotary printing techniques, have some significant drawbacks, such as manufacturing line speed limitations, quality issues, and reagent waste, to name just a few problems. On the other hand, inkjet printing of the reagent helps to remedy these issues. While some may have alluded to inkjet printing of the reagent, none have addressed the numerous issues associated with developing a reagent formulation that can be successfully printed using inkjet technology. The inventors have developed a commercially viable formulation for inkjet printing that does not significantly damage enzyme activity when the reagent is printed. The below discussed reagent ink formulation provides an accurate and uniform deposition of chemistry reagent on flexible circuitry using inkjet technology. This enables manufacturing of a biosensor with improved accuracy and precision than current techniques. Digital printing via inkjets enables a wide variety of printing patterns for a diverse product portfolio. It also enables printing of different reagent formulations at different positions on the same strip, or a dual layer printing system where different species can be laid one upon another. Different designs can be printed merely by changing the electronic file on a computer. No complex tooling change, machine set-up, cleaning validation, or machine stoppage is required.

The wet film produced by inkjet printing is made up by printing hundreds or thousands of very small (1 to 80 pico liter) drops at very high frequencies. This gives the ability to control the wet and dry film thickness in a very narrow range. This enables very uniform thin reagent films that are required for precision manufacturing, such as required in the production of accurate biosensors or test strips. Inkjet reagent dispensing is fast and accurate. Specific patterns can be printed in specific positions. Utilizing inkjet technology for applying chemical formulations for some current commercial test strips requiring patterns, a production line speed of 30 to 60 meters/minute can be achieved. This provides substantially faster production speeds than currently available using other deposition techniques, and inkjet technology can provide better precision and accuracy even at the highest production speeds. It is also conceivable to apply a first reagent on a substrate followed by a second different reagent on the same substrate on top of the first reagent or in near proximity of the first reagent. For example, for a test strip, an active reagent is applied as the first layer closest to the working and counter electrodes and a platelet separating polymer is applied as a second layer on top of this first layer. This can potentially improve the stability of the sensor.

The depositing of reagent by inkjet methods can be done on substantially flat substrates, substrates with electrodes on the surface, and substrates with other reagents on the surface. The substrate can be a polymer material such as polyester material (Melinex® polyester film). The surface of the polymer material can be untreated or treated, where treatments may include ablating or chemical rinse. The depositing of reagents may be into a well or depression on a substrate. A well may be formed by a second layer of material on the substrate in which a cut-out exists providing the sides of the well and forming the shape of the well where the reagent is to be deposited on to the substrate.

### Reagent Ink Formulation

When developing the reagent ink, a number of significant factors and issues were considered. The enzyme activity in the reagent ink needed to not be adversely affected by the inkjet printing process and/or the formulation of the ink. It was discovered that the enzymes were able to withstand the shear produced by the inkjet head without losing any activity.

Cracking or flaking of the reagent in the finished biosensor (i.e., reagent durability) was another concern. The developed reagent ink formulation was able to last 180 days before cracking started (under desiccant conditions, without flexing the strip). Early-development stage ink formulations cracked after only a few days. It was discovered that particle size helped to address this cracking issue. Nano-sized silica particles were incorporated into the ink, and the nano-sized silica particles showed an effect on how the dry film cracks, resulting in smaller cracks. The nano-sized silica also prevented flaking if cracking occurred, and it further affected hydration of the dry reagent film.

It was found that several factors affected the printed reagent film thickness and uniformity. One of those was the rheological properties of the reagent ink. The rheology requirements are very different than those for traditional slot-die coating (see, e.g., U.S. Pat. No. 7,749,437) and screen printing techniques. Specifically, ink printing requires very high shear thinning and has to be very accurate in order to meet reagent layer requirements. Further complicating matters is that the rheology requirements depend on the type of inkjet printing technology used. For example, bubble thermal jet printers require 1-3 cP viscosity, whereas piezo-electric printer need a viscosity of about 6-12 cP.

Surfactants in the ink formulation was another variable that was found to affect reagent film formation. While many types of surfactants will work in general for most inkjet printing needs, the incorporation of ionic surfactants was found to be undesirable because ionic surfactants damage enzyme activity. Within the group of non-ionic surfactant options, it was discovered that there were incompatibility issues with other components of the ink. Surprisingly, it was discovered that that the choice of surfactant had an effect on rheology. Some surfactants had an effect depending on concentration in the ink. Surfactants were selected with no (or little) effect in order to avoid having to account for the rheology effects. Surfactant effectiveness on reducing surface tension was also an issue, especially for the wetting properties when printing the ink. It was found that if the surface tension was too high, then printed dots of reagent ink would not mix properly, and if the surface tension was too low, then the reagent film would spread further than desired, which in turn would hurt line quality for the dried reagent film. As a result, surfactants were selected that had no effect on rheology, that were effective at reducing surface tension, and that were non-ionic (i.e., compatible with the enzyme/mediator system).

It was also found that the polymers incorporated into the reagent ink not only affected durability of the dried reagent but also homogeneity of the reagent layer profile (i.e., flatness of the reagent layer). Inks with lower molecular weight polymers tended to crack easily. However, other issues were experienced with polymers having high molecular weights.

The base formulation of the ink can include one or more, but not limited to, the following:

plasticizers such as ethylene glycol (EG),

polymers, which may act as film formers and/or rheological modifiers, such as polyvinylpyrrolidone (PVP), carboxymethyl cellulose (CMC), polyvinylchlorides (such as Propiofan®), hydroxyethylcellulose (such as Natrosol® 250 LR and Natrosol® 250 M), poly(ethylene-oxide) (PEO), poly(2-ethyl-2-oxazoline) (such as Aquazo150), polyvinyl alcohol (PVA), hydrophobically modified non-ionic polyols (such as Acusol™ 880 and 882), acrylate-based emulsion copolymers (such as Alcogum® L-15);

colloidal silica dispersions (such as Snowtex C);

surfactants, such as polyethylene glycol ethers (Triton® X-100), lithium carboxylate anionic fluorosurfactant (Zonyl® FSA), tetramethyldecyne diols (Surfynol® 104E), isopropyl alcohol (IPA) and propylene glycol;

solvents, such as 1-octanol, isopropanol (IPA), water;

buffers, such as phosphate, 1,4-piperazine bis(2-ethanosulfonic acid) (PIPES);

ionic strength modifiers, such as KCl, NaCl; and

pH modifiers such as KOH.

Reactive materials are then added to the base formulation to produce the final formulation to use in the production of the desired devices. The reactive materials are selected based on the type of device to be made. Reactive materials can include, but are not limited to, one or more of the following:

enzymes, such as, glucose dehydrogenase, glucose dye oxidoreductase, glucose oxidase and other oxidases or dehydrogenases such as for lactate or cholesterol determination, esterases etc.;

proteins, such as enzymes, bovine serum albumin;

co-factors (bound or unbound) for enzymes, such as NAD, NADH, PQQ, FAD;

mediators, such as ferricyanide, ruthenium hexamine, osmium complexes, or alternatively mediator-precursors such as nitrosoanilines;

stabilizers, such as trehalose, sodium succinate;

inorganic ions, such as Na+Cl—, K+Cl—;

indicators; and

dyes.

The reactive materials may include other chemical or reagents as necessary for the particular analysis that is to be done. Table 1 is a partial list of some combinations of reactive materials that could be included in a reagent ink formulation. The components in Table 1 list only the main reactants and do not include materials such as stabilizers (i.e., saccharides), ionic strength, or pH modifiers (KCl, or KOH) that may be found in the complete formulation of the reactive material that would be known to one in the art.

TABLE 1
A partial list of some analytes, enzymes, and mediators that can be used to measure the
levels of particular analytes.

| | | Mediator | |
|---|---|---|---|
| Analyte | Enzymes | (Oxidized Form) | Additional Mediator |
| Glucose | Glucose Dehydrogenase | Ferricyanide | |
| | and Diaphorase | | |
| Glucose | Glucose-Dehydrogenase | Ferricyanide | |
| | (Quinoprotein) | | |
| Cholesterol | Cholesterol Esterase and | Ferricyanide | 2,6-Dimethyl-1,4- |
| | Cholesterol Oxidase | | Benzoquinone |
| | | | 2,5-Dichloro-1,4- |
| | | | Benzoquinone or |
| | | | Phenazine Ethosulfate |
| HDL | Cholesterol Esterase and | Ferricyanide | 2,6-Dimethyl-1,4- |
| Cholesterol | Cholesterol Oxidase | | Benzoquinone |
| | | | 2,5-Dichloro-1,4- |
| | | | Benzoquinone or |
| | | | Phenazine Ethosulfate |
| Triglycerides | Lipoprotein Lipase, | Ferricyanide or | Phenazine Methosulfate |
| | Glycerol Kinase and | Phenazine | |
| | Glycerol-3-Phosphate | Ethosulfate | |
| | Oxidase | | |
| Lactate | Lactate Oxidase | Ferricyanide | 2,6-Dichloro-1,4- |
| | | | Benzoquinone |
| Lactate | Lactate Dehydrogenase | Ferricyanide | |
| | and Diaphorase | Phenazine | |
| | | Ethosulfate, or | |
| | | Phenazine | |
| | | Methosulfate | |
| Lactate | Diaphorase | Ferricyanide | Phenazine Ethosulfate, or |
| Dehydrogenase | | | Phenazine Methosulfate |
| Pyruvate | Pyruvate Oxidase | Ferricyanide | |
| Alcohol | Alcohol Oxidase | Phenylenediamine | |
| Bilirubin | Bilirubin Oxidase | 1-Methoxy- | |
| | | Phenazine | |
| | | Methosulfate | |
| Uric Acid | Uricase | Ferricyanide | |

In some of the examples shown in Table 1, at least one additional enzyme is used as a reaction catalyst. Also, some of the examples shown in Table 1 may utilize an additional mediator, which facilitates electron transfer to the oxidised form of the mediator. The additional mediator may be provided to the reagent in a lesser amount than the oxidized form of the mediator. While the above assays are described, it is contemplated that current, charge, impedance, conductance, potential, or other electrochemically indicated property of the sample might be accurately correlated to the concentration of the analyte in the sample with an electrochemical biosensor in accordance with this disclosure.

The general physical characteristics of material that can be dispensed from an inkjet printhead are given in Table 2. Formulations of the various components of the reagent ink are adjusted to provide physical characteristics that fall within the parameters set out in Table 2 to produce a reagent ink whose use through inkjet technology can generate acceptable results in making devices.

TABLE 2
General properties used in formulating a reagent ink

| Property | Design Constraints |
|---|---|
| Viscosity | 6 to 12 cP |
| Surface Tension | 28 to 33 dyne/cm |
| Molecular Weight | less than 90 kD; less than 50 kD better |
| Drop Formation | single, well defined drops; reproducible rate of |
| | formation |
| Film Thickness | 3 to 6 μm |
| Film Formation | uniform, homogeneous flat dry film |
| Dry Film Properties | Flexible; non-tacky; not susceptible to cracking or |
| | flaking |
| Activity | enzyme activity should not be affected or |
| | compromised by ink components |

Printing trials were performed using this ink using an Omnidot 760 GS8 printhead. A wide range of printing parameters (waveform, voltage offset, print frequency) were tested to apply the various ink formulations. Methods of using inkjet technology are also described in this disclosure. The density of printing or applying reagent to a substrate, measured in dots per inch (DPI), can be varied by adjusting the angle of the printhead with respect to the direction of motion between the printhead and the substrate. The printhead can be moved along a stationary substrate, the substrate can be moved along a stationary printhead, or both the printhead and substrate can be in motion. The number of printheads used to apply the reagent to the substrate can also be varied. The printheads can be aligned with each other, or can be offset from each other to provide better coverage of the substrate (increasing effective DPI). Increasing the number of printheads used to apply the reagent is used to increase the production speed of applying the reagent to the substrate.

### EXAMPLE 1

Several different polymers (film formers) were investigated by adding them to an existing composition of commercial interest to determine if an acceptable material for use with inkjet technology could be produced. The polymers investigated included Natrosol 250 LR, polyvinylpyrrolidone (PVP) K25 and K30, Aquazol 50, and Snowtex C. Natrosol 250 LR has already been used as a replacement material in biosensor formulations and has been shown to be inkjet printable. It should, however, be noted that Natrosol 250 LR has an estimated molecular weight of 90 kD, which is typically in the maximum region that is usually inkjet printable. Both PVP K25 and K30 are film formers which are conventionally and regularly used in inkjet printing. Aquazol 50 is a poly(2-ethyl-2-oxazoline) with molecular weight 50 kD which has good adhesive and film forming properties. Finally Snowtex C is a colloidal silica dispersion with particle size 10-20 nm at a concentration of 20%. The purpose of including silica is to aid with the “pinning” of the film to avoid the coffee stain effect seen in the previous ink formulations.

TABLE 3
Reagent ink formulated for use in an inkjet printhead produced by
modifying a commercial formulation

| Component | Wt % |
|---|---|
| Natrosol 250 LR | 0.452 |
| PVP K30 | 0.452 |
| Snowtex C | 0.603 |
| cPES | 0.118 |
| Gluc-DH | 3.791 |
| Nitrosoaniline) | 0.658 |
| NAD | 1.316 |
| Potassium chloride | 0.887 |
| Buffer solution | 89.666 |
| 1-octanol | 0.098 |
| IPA | 1.959 |
| Potassium hydroxide solution (5N) | + amount to adjust pH |

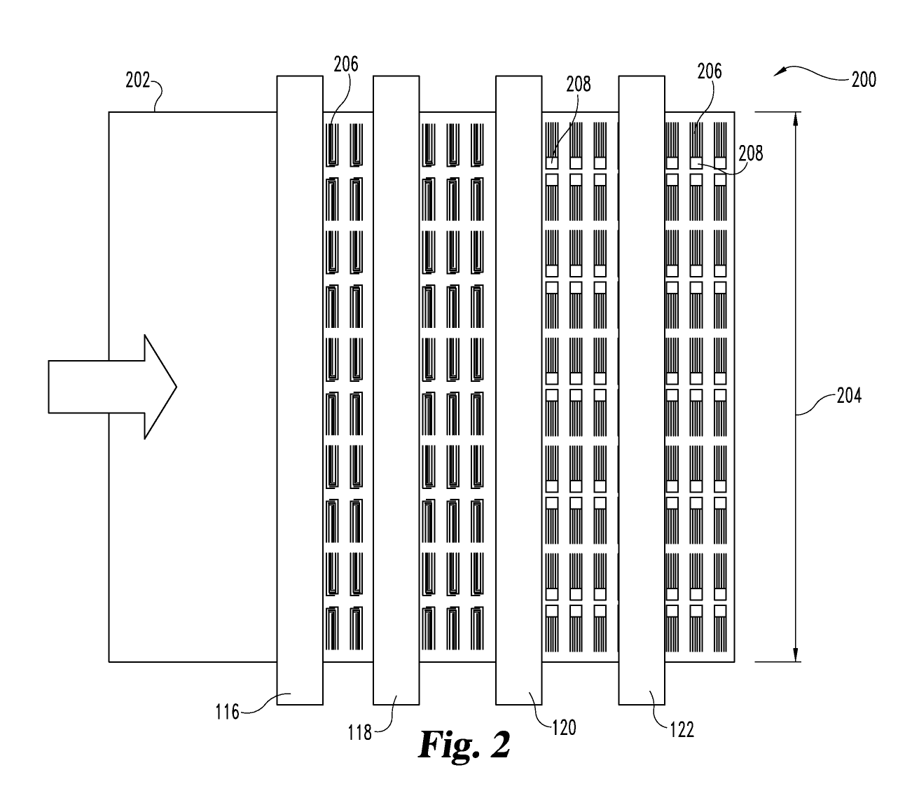

Samples were produced using this reagent ink formulation by printing strips in a range of resolutions (360×360, 720×720 and 1080×1080 dpi) and then drying in an oven at 45° C. for 2 min. These samples were then examined using profilometry and analyzed for response using linearity test solutions. The first set of tests were performed on samples produced on incomplete sensor substrates (i.e., pre-attachment of capillary wells) while the second set of tests were performed on samples on which ink had been printed into the capillary wells (see FIG. 2). Compared to the samples produced using the Freedom-type formulations, these print samples are clearly more homogenous.

A large set of samples were produced at 720×720 dpi. Examination of these films 2 months later showed that the layer had physically altered, with cracks running throughout the once-smooth continuous films. The flaking, brittle nature of this aged film was clearly undesirable and will have to be improved on. This type of behavior is most likely attributable to the drying conditions together with the ratio of polymeric material (e.g. Natrosol 250 LR and PVP K30) to particulate material (e.g. active materials).

Electrical responses of the 720×720 dpi samples were tested using linearity test solutions with both fresh and 2-month old samples (see Table 4; note that different units were used for response reporting). Assuming that both sets of results are comparable, then a significant increase in the percent Coefficient of Variation (% CV, imprecision of the measurements) has occurred after a 2 month period. This is very likely linked to the cracking and flaking of the film. Modifying an existing reagent formulation to make it usable with inkjet technology produced a composition that printed well but lacked stability over time, so this was not acceptable for a commercial product.

TABLE 4
Linearity testing for 720 × 720 dpi samples

| | Fresh samples | | | 2-month old samples | | |
|---|---|---|---|---|---|---|
| | Average | | | Average | | |
| | (mg/dL) | Std Dev | % CV | (pA) | Std Dev | % CV |
| Test | 33.4 | 2.99 | 8.95 | 0.43 | 0.06 | 14.60 |
| Solution 1 | | | | | | |
| Test | 88.7 | 5.00 | 5.64 | 0.96 | 0.22 | 22.36 |
| Solution 2 | | | | | | |
| Test | 256.1 | 6.84 | 2.67 | 2.16 | 0.63 | 29.27 |
| Solution 3 | | | | | | |

### EXAMPLE 2

### Determine Surfactant Effectiveness

A reagent ink base formulation was developed through testing of various film formers with various surfactants. The film formers used were PVA 9-10k, PVA 30-50k, PVP K15, PVP K30, Aquazol 50, Alcogum L15, Alcoguard 5800, and Alcusol 882. Surfactants used in the development were Triton X-100, Zonyl FSA, Surfynol 104E, IPA, and propylene glycol. Various combination and various concentrations were produced. The effects of concentration on surface tension and rheology were measured. Results from the test demonstrated the effectiveness of the surfactants on surface tension of a 7.2% solution of PVA 9-10k. Zonyl FSA was found to be an effective surfactant for all the film formers used in the study except Alcoguard 5800.

### EXAMPLE 3

### Printability of Film Former Blends

Combinations of the polymers PVA 9-10k, PVA 30-50k, PVP K30, Aquazol 50, Alcogum L15, and Alcoguard 5800 with each other were blended with Zonyl FSA surfactant to produce a reagent ink base composition. The properties of these combinations that were studied included viscosity, surface tension, ease of printing setup, effect of waveform, effect of drive voltage, drop formation, printing reliability, film formation and film resilience. The results indicated that PVP K30+PVA 30-50k produced acceptable printed film thickness uniformity, PVA 9-10k produced a crack-resistant film and PVP K30+PVA 30-50k produced a film that delayed cracking. Aquazol 50 produced unacceptable printed film thickness uniformity and PVA 9-10K+PVA 30-50K produced a formulation that was unacceptable for printing.

### EXAMPLE 4

### Ink Formulation 1

A formulation of reagent ink was produced which included polymers PVP K30, PVA 9-10K and PVA 30-50K as film formers and ethylene glycol as a plasticizer. The ratio of PVP K30 to PVA 9-10K is in the range of about 50:50 to about 90:10 or about 60:40 to about 80:20, or preferably about 80:20. The composition includes about 0.5% PVA 30-50K and about 2% ethylene glycol. This formulation produced a reagent ink that is easy to set up for printing and reliable. The viscosity was measured as 12.1 cP, and the surface tension was measured as 20.7 dyne/cm. The reagent ink produced good dry film uniformity printing at 1080×1080 dpi to give a thickness of about 4 to 5 □m. The dry films do not crack upon aging, and the films are reactive and can generate a detectable signal when appropriate active reagents are included.

### Inkjet Manufacturing Process

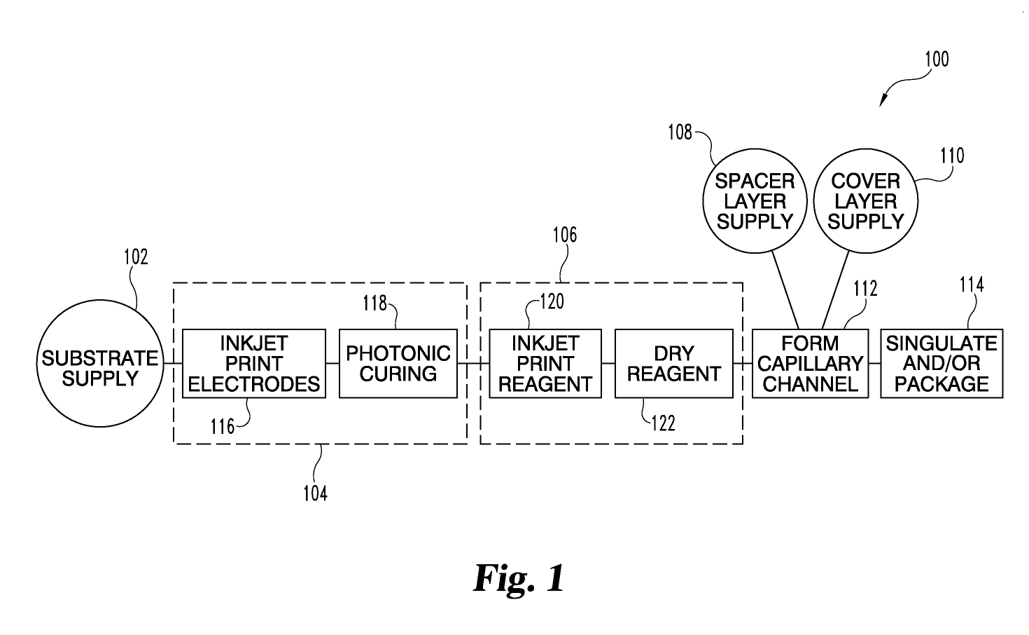

A technique for manufacturing a biosensor using inkjet printing techniques will now be described with reference to FIG. 1. FIG. 1 generally depicts a biosensor manufacturing system 100 for producing biosensors using inkjet printing techniques to deposit the electrodes and reagent onto the substrate. It should be noted that FIG. 1 only depicts a few of the general manufacturing stages, and it should be recognized that other stages, such as various cleaning, heating, cooling, and treating stages, can be incorporated into the system 100. Moreover, the technique will be described with reference to a roll-to-roll manufacturing process, but it is contemplated that other types of manufacturing processes could be used. The system 100 includes a substrate supply 102 that supplies the substrate upon the electrodes, reagent, and other components are layered in order to form the biosensor. In one example, the substrate supply 102 is in the form of a roll around which the substrate is wound, but it is contemplated that the substrate can be supplied in other manners. The system 100 further includes electrode 104 and reagent 106 formation stages in which the electrodes and reagent patterns are respectively formed on the substrate. To form the capillary channel and/or testing chamber in which the body fluid sample is deposited for analysis, a spacer layer that is supplied from a spacer layer supply 108 and a cover layer that is supplied from a cover layer supply 110 are sealed with the substrate at stage 112. In one example, the spacer layer 108 and cover layer 110 supplies are in the form of rolls or reels around which the material is wound, but it is envisioned that the spacer layer and cover layer material can be supplied in other manners. After the capillary channels are formed in stage 112, the substrate can be cut or otherwise singulated to form individual biosensors or test strips in stage 114. The now individualized test strips can packaged in conventional packaging for shipment to consumers. Alternatively or additionally, the biosensors in stage 114 can be packaged into multi-biosensor packaging, such as cassette tapes, cartridges, drums, and the like.

By using inkjet printing techniques to form both the electrodes and the reagent, the space occupied by the biosensor manufacturing system 100 is considerably smaller because the length of the line can be shortened. Moreover, compared to conventional drop deposition or slot-die coating techniques the inkjet printing techniques described herein facilitate the use of wider substrates, which in turn increases the production throughput. In addition, this all-inkjet manufacturing technique allows greater flexibility in the design of the biosensors as well as quick changeovers in biosensor types. In essence, given the inkjet printers are digitally controlled, they can be changed on the fly, that is, while system 100 is still producing biosensors. This ability to rapidly change parameters also allows feedback type controls for improving the overall product quality. In one example in the electrode formation stage 104, the electrodes are formed using an inkjet printing technique of the type described in U.S. patent application Ser. No. 12/862,262, filed Aug. 24, 2010, which is hereby incorporated by reference in its entirety.

Looking at FIG. 1, during the electrode formation stage 104, an electrode inkjet printer 116 forms an electrode pattern on the substrate, and a photonic curing machine 118 sinters the electrode pattern so that proper conductance of the electrodes is established. During the electrode formation stage 104, a reagent inkjet printer 120 prints the reagent onto one or more locations on the electrodes and/or substrate. The printed reagent can be air dried and/or dried via a reagent dryer 122. In one example, the reagent dryer is a conventional electric dryer that blows hot air across the substrate in order to dry the reagent, but it is contemplated that the reagent can be dried in other manners, such as via IR heats lamps and blowers, to name just a few examples. Again, it should be recognized that the reagent dryer 122 can be optional in certain circumstances such that the reagent is air dried. While there are a number of significant benefits of using an all inkjet manufacturing technique, it is contemplated that a hybrid approach can also be used in which the electrodes are formed using conventional means, such as for example by screen printing, laser ablation, etc., while the reagent is inkjet printed onto the electrodes and substrate.

FIG. 2 shows a top view of one example section of a biosensor manufacturing line 200 that utilizes an all inkjet printing approach. It should be emphasized that FIG. 2 shows just shows one exemplary section of the line 200 where the electrodes and reagent are formed via inkjet printing, and it should be appreciated that the line 200 can incorporate other equipment, such as cleaners and cutting equipment, to name just a few examples. A base substrate 202 is fed into the line 200, as is indicated by the arrow in FIG. 2, from the substrate supply 102 (FIG. 1). One of the many benefits of using inkjet printing for depositing reagent over conventional techniques, such as slot-die coating or screen printing, is that the width 204 of the base substrate 202 can be considerably larger. For instance, the substrate used in conventional reagent deposition techniques is typically limited to about 1 (one) foot wide, whereas the width 204 of the base substrate 202 using the inkjet printing technique described herein can be 5 (five) feet (60 inches) or even wider.

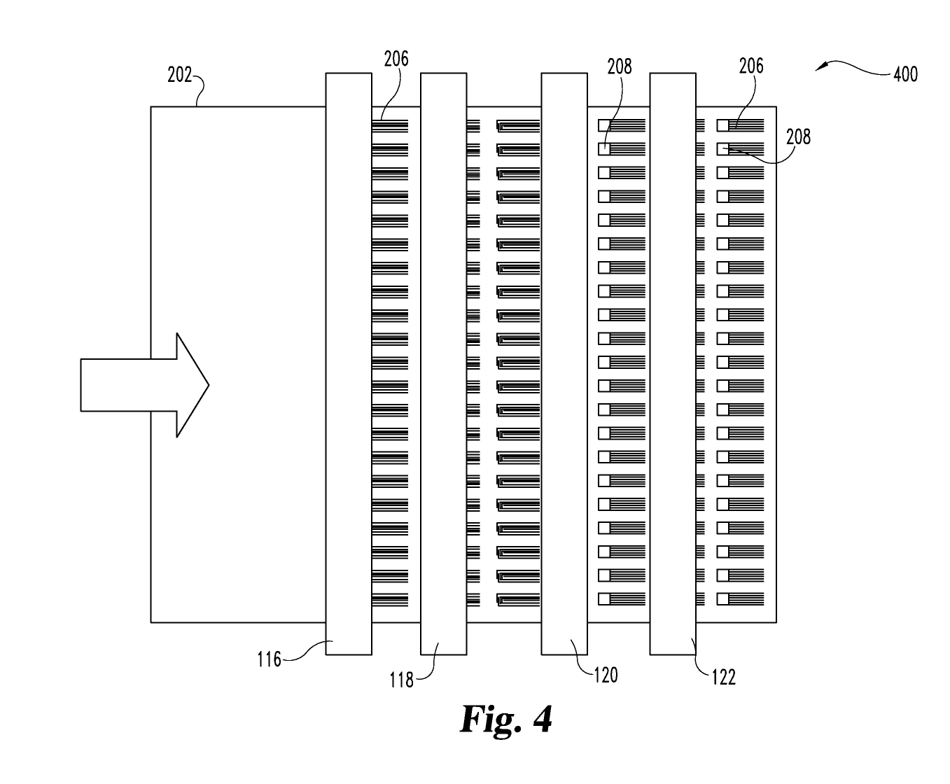

Once the base substrate 202 is supplied, electrodes 206 are inkjet printed with the electrode inkjet printer 116, and subsequently, the electrodes 206 are sintered via the photonic curing equipment 118. For a detailed description of forming the electrodes 206 using inkjet printing, please refer to U.S. patent application Ser. No. 12/862,262, filed Aug. 24, 2010, which is again hereby incorporated by reference in its entirety. In one example, the electrodes 206 are made of carbon, but in other examples, the electrodes 206 can be made from other types of conductive materials, such as silver, aluminum, ITO, gold, platinum, palladium, copper, and/or a combination of materials, to name just a few examples. The electrodes 206 shown in FIG. 2 generally extend in a direction that is transverse, and in this particular example perpendicular to, the direction in which the substrate 202 is fed, which is shown by the arrow in FIG. 2. As will be explained in greater detail below, the electrodes 206 can be oriented in other manners (see e.g., FIG. 4).

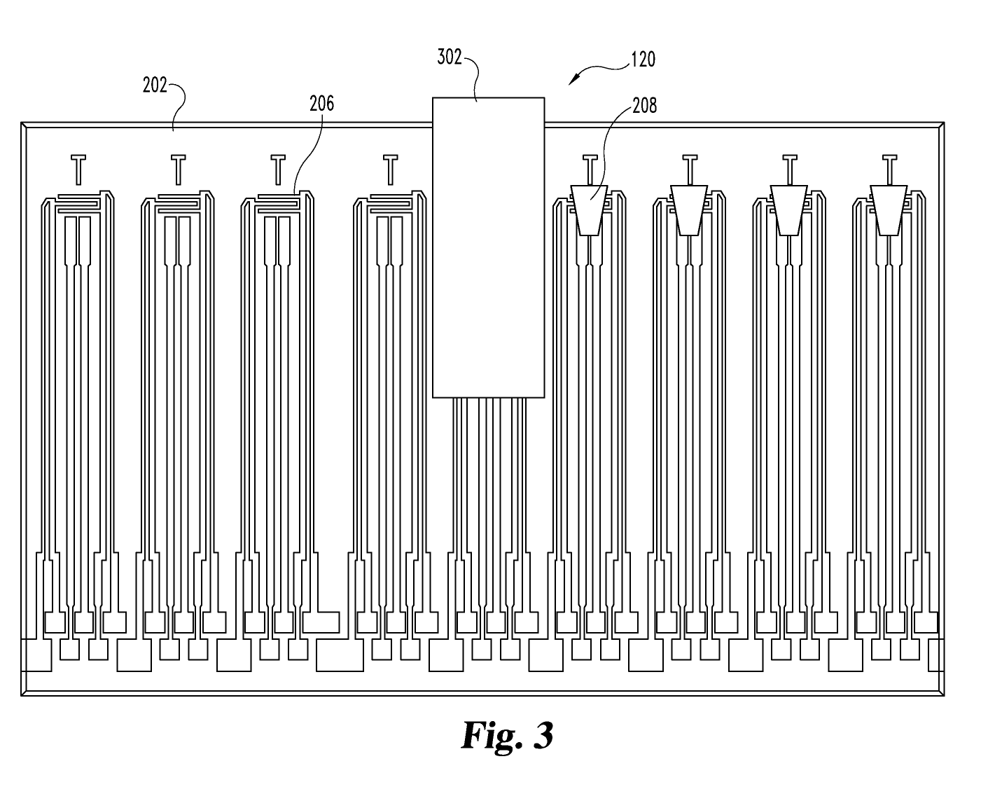

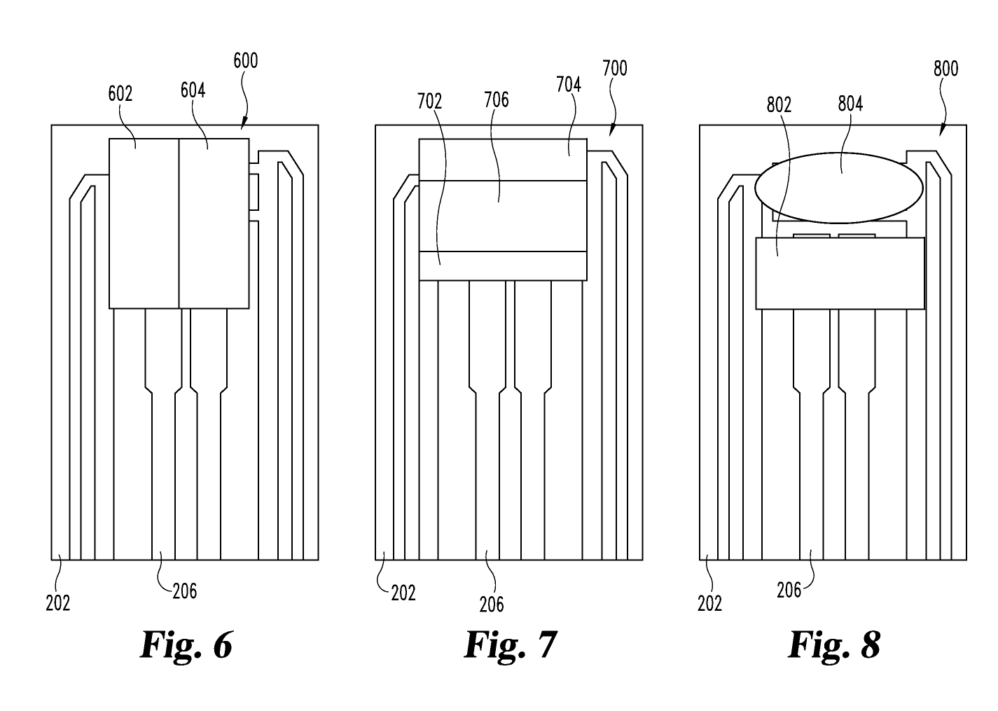

After the electrodes 206 are formed, the reagent inkjet printer 120 inkjet prints reagent 208 having the formulation described above over a portion of the electrodes 206 that form the analysis portion or chamber of the test strip. The reagent inkjet printer can print the reagent in a number of different manners such as through continuous or drop on demand techniques. FIG. 3 shows an enlarged view of a reagent inkjet printhead 302 of the reagent inkjet printer 120 printing the reagent 208 onto the base substrate 202 and electrodes 206. In the illustrated embodiment, the reagent printhead 302 is a piezoelectric type printhead. By using a piezoelectric type printhead or other acoustic type printheads, there is no or lower risk of thermal damage to the reagent as compared to thermal type inkjet printers. However, where the risk of thermal damage to the reagent is low and/or controllable, it is contemplated that thermal inkjet type printers can be used. The reagent inkjet printhead 302 can be a fixed or disposable type, depending on the requirements. The reagent inkjet printer 120 can have a single reagent printhead 302 to print all of the reagent or multiples printheads 302. When a single reagent printhead 302 is used, the reagent inkjet printhead 302 can span the entire width 204 of the base substrate 202 or the printhead 302 can be moveable so as to print across the entire width 204 of the base substrate 202. Likewise, when multiple reagent printheads 302 are used, the reagent printheads 302 can be fixed or moveable. In addition, the reagent printheads 302 can contain different reagent formulations and/or chemical compositions such that the printheads 302 are able to form different reagent layers and/or separate testing areas with different types of reagents (see e.g., FIGS. 6, 7, and 8). In one embodiment, the reagent printhead 302 is a Xaar Omnidot 760 GS8 printhead due to its low dead volume properties. However, it is contemplated that other types of printheads can be used, such as a Xaar 1001 printhead or those manufactured by Konica-Minolta, to name just a few examples.

Looking again at FIG. 2, to properly dry the reagent 208 to avoid issues, such as cracking of the reagent, coffee staining, thickness uniformity, and the like, the reagent 208 is dried with the reagent dryer 122. The reagent dryer 122 can incorporate multiple drying stages or can have a single stage. In another embodiment, the reagent dryer 122 can be eliminated such that the reagent is air dried. After drying, the base substrate 202 then proceeds to the capillary channel formation 112 and singulation/packaging 114, as are depicted in FIG. 1.

As mentioned before, the electrodes 206 and reagent 208 can be oriented in a different manner than is shown in FIG. 2. For example, FIG. 4 shows a biosensor manufacturing line 400 in which the electrodes 206 are oriented in a direction that is generally parallel to the direction in which the base substrate 202 is fed, as is shown by the arrow. In still yet other examples, the electrodes 206 and reagent 208 can be oriented generally diagonal to the feed direction of the base substrate 202. Due to the greater flexibility of the inkjet printing, the electrodes 206 and reagent 208 can be oriented in different directions relative to one another on the same substrate 202 in order, for example, to improve printing density as well as minimize waste.

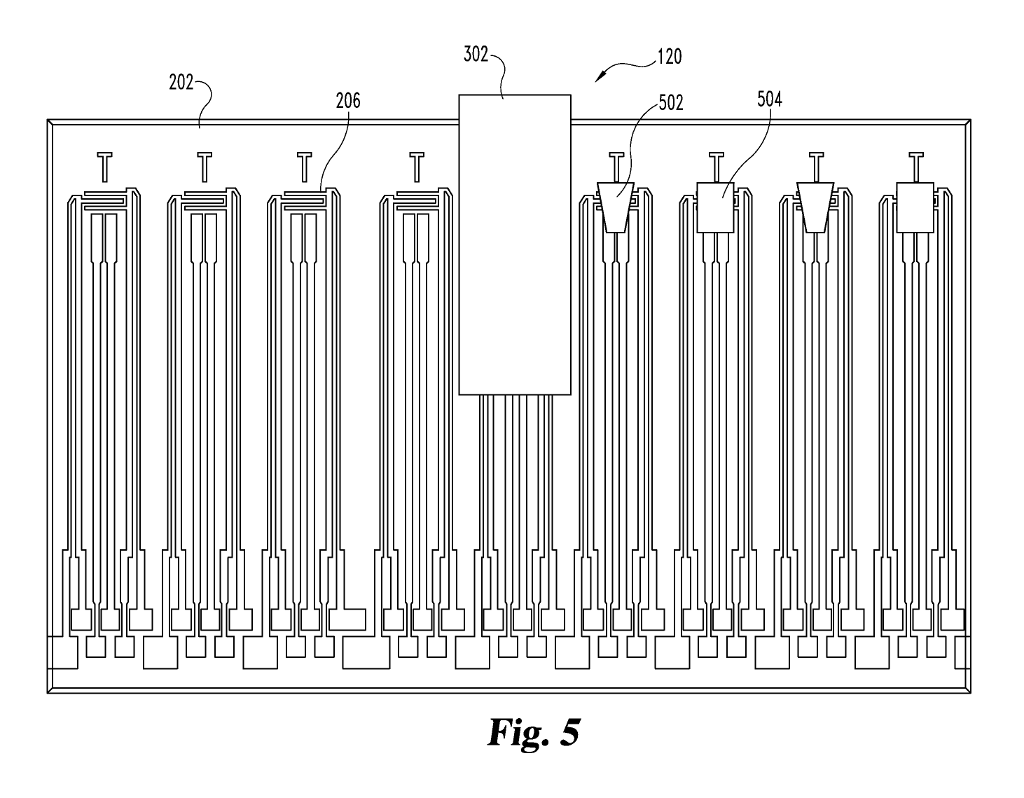

Again, due to the digital nature of inkjet printing, the biosensor designs can be quickly changed over, even while the line is still operating. For instance, FIG. 5 shows one embodiment in which the reagent inkjet printhead 302 prints reagent with different patterns or shapes 502, 504. In the illustrated embodiment, the first reagent pattern 502 has a trapezoidal shape, and the second reagent pattern 504 has a rectangular shape, but the reagent patterns 502, 504 can be shaped differently in other embodiments. The different reagent patterns 502, 504 can be used to produce different biosensor types on the same line. The reagent patterns 502, 504 can have the same chemical composition or be formulated differently to, for example, detect different analytes. For instance, this approach can also produce a dual use biosensor that tests for the similar or different analytes. In the finished biosensor, the reagent patterns 502, 504 can be oriented in a coplanar arrangement or located on different sides. As an example, the base substrate 202 can be folded such that the different reagent patterns 502, 504 can be on different sides so as to create a double-sided biosensor. In one particular embodiment, the double-sided biosensor can be used to simultaneously measure both glucose and ketone levels. Although one reagent printhead is shown, it should be recognized that multiple printheads can be used to increase the line speed and/or to print different reagent patterns 502, 504 that have different chemical compositions.

To improve testing accuracy, reagents or other layers with different chemical compositions can be printed in the same general vicinity of one another. For instance, FIG. 6 shows an enlarged view of a reagent inkjet printing pattern 600 according to another embodiment. As shown, the reagent pattern includes first 602 and second 602 reagents with different chemical compositions printed in a side-by-side orientation over the base substrate 202 and electrodes 206. In this embodiment, the first reagent 602 has the same formulation as the second reagent 604 with the exception that the first reagent 602 does not include any enzymes. In essence, the first reagent 602 is used as a control in order to detect and/or compensate for environmental abuse that may have adversely affected the enzymes in the second reagent 604. FIG. 7 illustrates another reagent inkjet printing pattern 700 in which different first 702 and second 704 reagents are inkjet printed in an overlapping manner to form an overlap section 706. In the overlap section 706, the first 702 and second 704 reagents can form distinct layers or mix together to create a mixture of the two reagents 702, 704. FIG. 8 depicts still yet another inkjet reagent pattern 800 to show that first 802 and second 804 reagents not only can have different chemical compositions and/or properties but also can be shaped differently. FIG. 8 in addition shows that the reagents 802, 804 can be spaced apart so as to not contact one another. As can be seen, one of the many benefits of using inkjet printing is the ability to have bare electrode sections as well. Of course, it is contemplated that other reagent patterns besides the ones illustrated herein are possible.

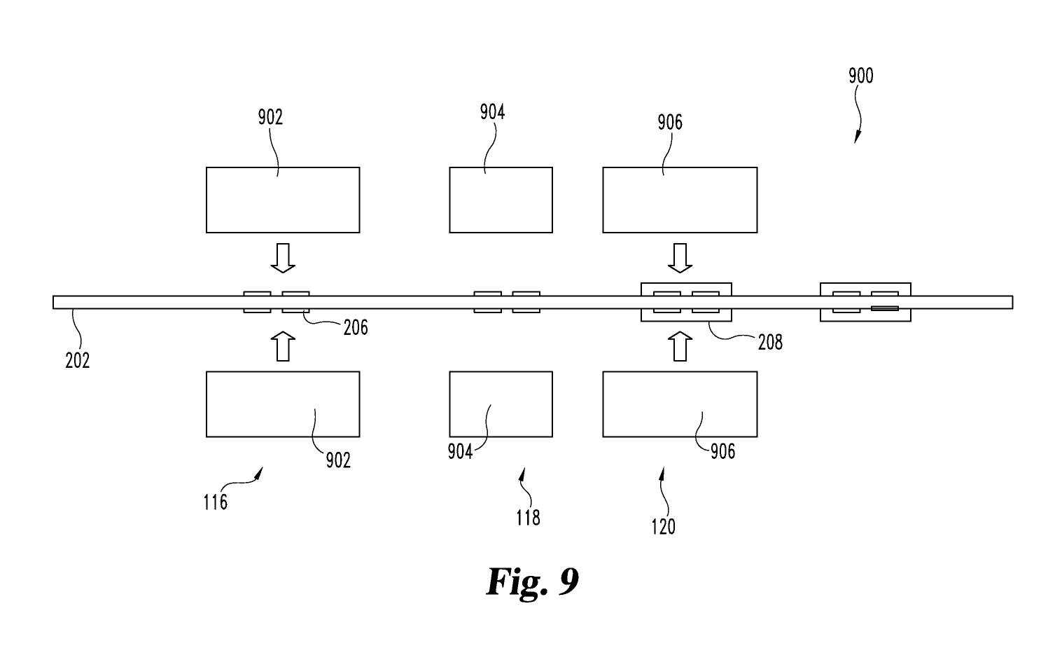

In addition, the inkjet printing techniques described herein allow for greater flexibility in biosensor design. For example, FIG. 9 illustrates a section of a double-sided biosensor manufacturing system 900 that prints electrodes 206 and reagent 208 on opposing sides of the base substrate 202. At the electrode inkjet printer 116, the system has two (or more) electrode printheads 902 facing the opposing sides of the base substrate 202 so that the electrodes 206 are inkjet printed on the opposing sides. Downstream from the dual electrode inkjet printheads 902, the photonic curing machine 118 has opposing emitters 904 that sinter the electrodes 206. As can be seen, the reagent inkjet printer 120 has two (or more) reagent printheads 906 that face the opposing sides of the base substrate 202 so as to spray the reagent 208 onto the opposing sides of the base substrate 202. Subsequently, the base substrate 202 can be processed in the manner described (i.e., reagent dried, form the capillary channel, package, etc.). While the various printheads are aligned with one another so that both sides are printed simultaneously, it should be appreciated that the various printhead and/or emitter pairs can be offset so that the various sides can be printed in a sequential fashion. For example, the reagent printheads 906 can be offset so that one side of the base substrate is printed with reagent 208 before the other side. In another example, the base substrate 202 can be flipped and ran through the same machine twice so that the electrodes 206 and reagent 208 are printed on both sides even when the machine only has one printhead of each type. Vias that connect the electrodes 206 on both sides of the base substrate 202 can also be formed using inkjet printing techniques and/or in other manners.

One of the many benefits of the inkjet printing techniques described herein is the ability to precisely pattern the reagent 208. The thickness and size of the reagent 208 can be tightly controlled which in turn improves the accuracy of the test results. In one embodiment, the inkjet printing technique allows the thickness of the reagent to be tightly controlled within a 5% tolerance. This ability to tightly control reagent patterning also helps to improve manufacturing yields, especially when the capillary channel is formed. If the reagent pattern is not tightly controlled, such as with traditional reagent deposition techniques, the reagent 208 can flow or wick over to where the spacer layer is attached to the base substrate 202,which in turn can be problematic for securing the spacer layer to the base substrate 202. The reagent 208 may interfere with adhesion if an adhesive is used to glue the layers together, or may interfere with laser welding the layers together. Another concern is that the excess reagent can also swell under the spacer. Again, the precise nature of inkjet printing the reagent helps to mitigate these issues.

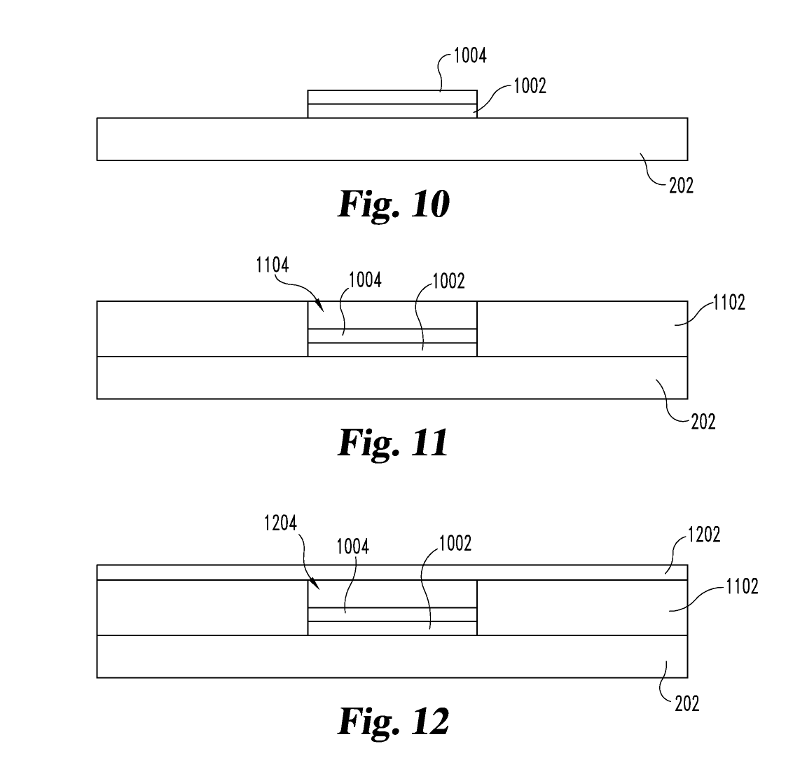

FIGS. 10, 11, and 12 illustrate this particular benefit of reagent inkjet printing when the capillary channel is formed in stage 112 (FIG. 1). FIG. 10 shows how the reagent can be precisely patterned such that it does not interfere with the subsequent steps. In the embodiment illustrated in FIG. 10, a first reagent layer 1002 and a second reagent layer 1004 are inkjet printed onto the substrate. The reagent layers 1002, 1004 can have the same formulation or a different formulation. For example, the second layer 1004 may not contain any reagent at all, but the second layer 1004 may act as a protective cover for the first reagent layer 1002 and/or act to filter red blood cells so as to minimize the hematocrit effect. Although two reagent layers 1002, 1004 are depicted in FIG. 10, it should be appreciated that one or more than two reagent layers can be inkjet printed onto the substrate 202 and over a section of the electrodes 206. For example, it is envisioned that a three-layer approach can be used in which the middle layer acts as a barrier so as to separate the other layers which are incompatible with one another. Looking at FIG. 11, a spacer layer 1102 with a capillary cutout 1104, which helps to form the capillary channel, is sealed with the base substrate in any number of different manners, such as with an adhesive and/or laser welding, to name just a few examples. Again, the precise printing control provided by inkjet printing helps to ensure that the reagent layers 1002, 1004 precisely match the capillary cutout so that the reagent does not interfere with the sealing of the spacer layer 1102 to the base substrate 202. As illustrated in FIG. 12, a cover layer or film 1202 is sealed to the spacer layer 1102 to form a capillary channel 1204. The cover layer 1202 can be sealed to the spacer layer 1102 through an adhesive, laser welded, and in other manners known in the art.

It should be recognized that the described and illustrated manufacturing stages can occur in different orders and/or hybrids of the various techniques are also contemplated. For example, the reagent 208 can be applied after the spacer layer 1102 is sealed to the substrate 202. Alternatively, the first reagent layer 1002 in FIG. 10 is inkjet printed before the spacer layer 1102 (FIG. 11) is applied, but the second reagent layer 1004 is inkjet printed into the capillary cutout 1104 after the spacer layer 1102 is secured to the base substrate 202. The various stages can also be split up so that only partial structures are formed. For example, the section of the electrodes 206 that is located underneath the reagent 208 are printed before the reagent 208 is applied, but the rest of the electrode sections are not printed until after the reagent 208 is printed. This can be helpful when the electrodes 206 are made from two different materials. The flexibility of inkjet printing also allows the electrodes to be structured in unconventional ways but still be able to function. For instance, inkjet printing allows the electrodes 206 to be printed in a sandwich like manner between the first 1002 and second 1004 reagents layers by printing the electrodes 206 after the first reagent layer 1002 is printed but before the second reagent layer 1004 is printed. In still yet another unconventional manner, it is contemplated that all or part of the electrodes 206 can be printed on top of the reagent 208 such that all or part of the reagent 208 is sandwiched between the base substrate 202 and the electrodes 206. It is also envisioned that all or part of the base substrate 202 can be cut (stage 114 in FIG. 1) before the electrodes 206 and/or reagent 208 are printed.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A method of manufacturing a biosensor, comprising:
forming an electrode on a substrate; and
inkjet printing a reagent over at least a portion of the electrode on the substrate.

2. The method of claim 1, wherein said forming the electrode includes inkjet printing the electrode onto the substrate.

3. The method of claim 2, further comprising:
photonically curing or sintering the electrode on the substrate.

4. The method of claim 1, wherein said inkjet printing the reagent includes:
inkjet printing a first layer; and
inkjet printing a second layer.

5. The method of claim 4, further comprising:
wherein the first layer includes an enzyme and a mediator;
wherein said inkjet printing the second layer includes inkjet printing the second layer over the first layer; and
wherein the second layer acts as a protective cover to protect the first layer.

6. The method of claim 4, further comprising:
inkjet printing a third layer over the second layer;
wherein the first layer and the third layer are incompatible; and
wherein the second layer acts as a barrier to separate the first layer and the second layer.

7. The method of claim 4, wherein the first layer and the second layer are spaced apart at separate locations on the substrate.

8. The method of claim 4, wherein said inkjet printing the second layer includes inkjet printing the second layer on top of the first layer.

9. The method of claim 4, wherein the first layer and the second layer have different shapes.

10. The method of claim 4, further comprising:
wherein said forming the electrode on the substrate includes
forming a first electrode pattern on a first side of the substrate, and
forming a second electrode pattern on a second side of the substrate that is opposite the first side of the substrate;
wherein said inkjet printing the first layer includes inkjet printing the first layer on the first side of the substrate; and
wherein said inkjet printing the second layer includes inkjet printing the second layer on the second side of the substrate.

11. The method of claim 4, further comprising:
securing a spacer layer to the substrate after said inkjet printing the first layer; and
wherein said inkjet printing the second layer occurs after said securing the spacer layer.

12. The method of claim 8, wherein said inkjet printing the reagent includes inkjet printing at least third, fourth and fifth layers, and wherein the third layer is on top of the second layer, the fourth layer is on top of the third layer, and the fifth layer is on top of the fourth layer.

13. The method of claim 1, wherein the substrate is at least 60 inches wide.

14. The method of claim 1, further comprising:
drying the reagent with a drying mechanism.

15. The method of claim 1, further comprising:
securing a spacer layer to the substrate after said inkjet printing the reagent; and
securing a cover layer to the spacer layer to form a capillary channel.

16. The method of claim 15, further comprising:
supplying the substrate with a substrate reel;
supplying the spacer layer with a spacer layer reel; and
supplying the cover layer with a cover layer reel.

17. The method of claim 1, further comprising:
moving the substrate at a line speed of at least 3 meters per minute during said inkjet printing the reagent.

18. The method of claim 1, further comprising:
inkjet printing a second portion of the electrode after said inkjet printing the reagent.

19. A biosensor, comprising:
a substrate;
an electrode pattern formed on the substrate;
a first reagent layer covering at least a portion of the electrode pattern;
a second reagent layer covering at least a portion of the first layer;
a third reagent layer covering at least a portion of the second layer;
wherein the third reagent layer is incompatible with the first reagent layer; and
wherein the second reagent layer acts as a barrier to separate the first reagent layer from the second reagent layer.

20. The biosensor of claim 19, further comprising:
a spacer layer secured to the substrate; and
a cover layer covering the spacer layer to form a capillary channel.
